(19) United States
(12) Patent Application Publication
(10) Pub. No.: US 2025/0344726 A1
(43) Pub. Date: Nov. 13, 2025

(54) SOLID STATE FERMENTATION FOR THE PRODUCTION OF NUTRIENT DENSE INGREDIENTS USING DEFINED COMBINATIONS OF FUNGAL AND BACTERIAL STRAINS

(71) Applicant: Mycsology Foods Inc., Princeton, NJ (US)

(72) Inventor: Yekaterina Tarasova, Medford, MA (US)

(73) Assignee: Mycsology Foods Inc., Princeton, NJ (US)

(21) Appl. No.: 19/203,472

(22) Filed: May 9, 2025

Related U.S. Application Data
(60) Provisional application No. 63/644,652, filed on May 9, 2024.

(51) Int. Cl. A23L 11/50; A21D 2/36; A21D 13/045; A23J 3/14; A23L 7/10; A23L 7/104; A23L 11/00; C12N 1/14; C12N 1/20

(52) U.S. Cl. CPC A23L 11/50; A21D 2/362; A21D 13/045; A23J 3/14; A23L 7/104; A23L 7/198; A23L 11/05; C12N 1/14; C12N 1/20

(57) ABSTRACT

A method to produce a nutrient-dense and flavorful fermented shelf stable food product are described. The method comprises using defined fungal and bacterial strains in a solid-state fermentation process of legumes, grains and/or seeds. Following fermentation under specified environmental conditions, minimal downstream processing is performed to preserve the flavors generated via fermentation. This resulting product can be used to make nutritious, highly flavorful food products.

This application is a U.S. Non provisional patent application which claims priority under 35 USC 119(e) to US Provisional Application No. 63/644,652 filed on May 9, 2024, the entire contents of which are incorporated by reference in its entirety.

### FIELD OF INVENTION

The present invention relates to a method of transforming plant material to produce nutritionally dense ingredients and foods by solid state fermentation. M ore particularly, the invention relates to the use of defined microbial species to achieve improved flavor and nutrient profiles of resulting ingredients suitable for use in a variety of food products.

### BACKGROUND OF THE INVENTION

Consumption of nutrient-poor, excessively processed foods has been increasingly linked to negative health outcomes such as diabetes, with studies suggesting a strong association between their intake and the development of disease. These highly processed foods often contain high levels of refined carbohydrates, unhealthy fats, additives, and exogenous flavors while lacking essential nutrients, fiber, and antioxidants. Their excessive consumption can lead to weight gain, insulin resistance, inflammation, and ultimately increase the risk of type II diabetes and other chronic diseases (Lane et al. 2024). Despite their poor nutritional qualities, these foods such as snack bars, crackers, and chips continue to be widely consumed due to their convenience, long shelf life, and low cost.

Fermentation of plant and animal products has been used for thousands of years mainly as a preservation method, and to achieve diverse flavors and textures as is seen in the diversity of available cheeses around the world today. Additionally, the presence of microbes themselves as live and active cultures, or probiotics, confers health benefits due to their interaction with the human microbiome and the immune system. Recently, even dead or non-viable microbial cells or cellular components have been shown to confer health benefits on the host, similar to those attributed to live probiotics (Siciliano et al. 2021).

It is known that both fungi and bacteria produce secondary metabolites that impart flavor, alter texture, and contribute to the overall mouthfeel and eating experience. While bacterial fermentation is more widespread, fungal fermentation also offers unique health benefits. Foods like tempeh, traditionally fermented with one type of fungus R. oligosporus, are high in protein, vitamins and micronutrients (Teoh et al. 2024). Other examples of fermentation with fungal strains relate to the use of A. oryzae to make sake and soy sauce, the latter being enriched in umami flavor compounds produced by the fungus.

Despite these many advantages, widespread consumption of foods fermented with fungal strains remains limited, possibly due to low consumer acceptance such as undesirable texture and taste resulting from the fermentation conditions used in its production. For example, while many descriptions for tempeh production exist, industrial tempeh production predominantly uses soy as the main substrate and is most frequently inoculated with only a single fungal strain.

Although it is clear that the consumption of fermented foods confers many health benefits, their worldwide consumption is limited due to cold chain requirements, or their often unappealing taste. Thus there is a need to produce similarly nutritionally dense, fermented foods that are stable at room temperature and are either ready to consume or able to be incorporated in a variety of other products.

It is with these drawbacks in mind that the present invention was created.

### BRIEF SUMMARY OF THE INVENTION

The inventor's insight is that combinations of bacterial and fungal strains can be used on various legume, seed and grain substrates under very specific environmental conditions in order to create fermentation products with more desirable flavor and texture characteristics. Post fermentation, minimal processing of the fermented material is accomplished resulting in nutrient-dense, paraprobiotic-containing ingredient. In this way, fermentation's benefits can be made more widely accessible.

Reported herein is a method for the production of shelf stable, paraprobiotic, nutrient-dense, nutrient balanced ingredients. This method utilizes defined bacterial and fungal strains to ferment legumes, grains and/or seed plant materials. The approach aims to minimize post-fermentation processing to preserve the developed flavor and nutrient profiles of the fermented ingredients, facilitating the production of foods that are both nutritious and delicious.

The present invention relates to compositions of legumes, seeds, and/or grains that are fermented with defined strains of fungi and bacteria to produce nutrient dense flour blends that can be used as ingredients in different food products.

The fermentation parameters of substrate inputs, and their form factor; inoculum type, ratio and amount; and fermentation conditions such as pH, moisture and temperature; are all controlled to result in specific nutrient and organoleptic profiles. Post fermentation, the resulting products are mildly processed by heating, caking, dehydrating, and milling, either alone or in combination to produce shelf stable enriched products as compared to the original starting plant material inputs of legumes, seeds and/or grains.

### BRIEF DESCRIPTION OF THE DRAWINGS

FIG. 1 shows a fermented product made via inoculation with one fungal and one bacterial strain and fermented at a constant temperature of 28° C. for 30 hours (A) followed by drying and milling into a flour (B).

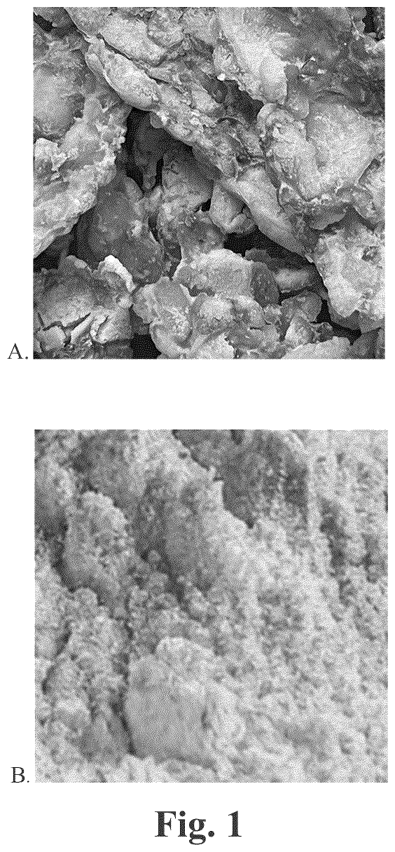

FIG. 2 shows a representative batch of fermentation product that is turned into a flour and used to make a finished food product such as crackers.

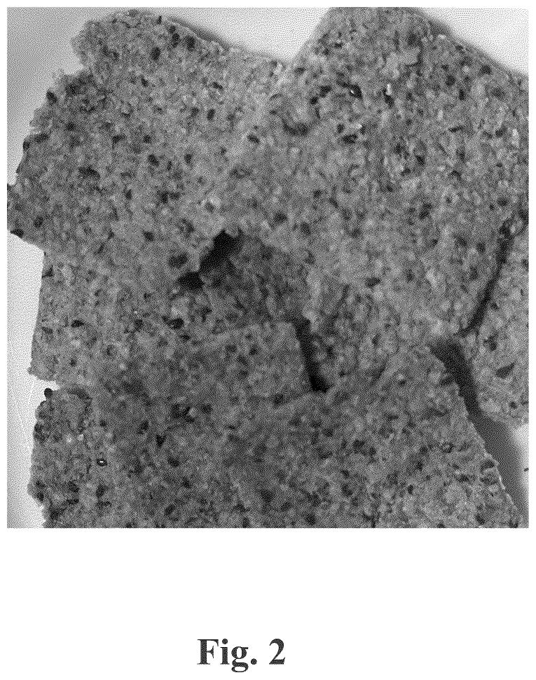

FIG. 3 shows an unfermented (i.e., C=control) chickpea sample from a top down view (upper left figure) and a side view (lower left figure) and the top down view (upper right figure) and the side view (bottom right figure) of the fermented (i.e., F) version adjacent to the control.

FIG. 4 shows fermented chickpea flour in focaccia dough at amounts of 0%, 10%, and 25% (from left to right) respectively.

FIG. 5 shows different fermented amounts of flours that display differences in particle size and color.

FIG. 6 shows a flowchart of the process to generate a fermented flour.

### DETAILED DESCRIPTION OF THE INVENTION

In some embodiments, the method of producing nutrient-dense flour blend comprises using one or more legumes, grains or seeds as substrate inputs into the fermentation, alone or in any combination thereof.

The legume substrates may be selected from the following list: Adzuki bean, alfalfa, Bambara groundnut, black gram, black-eyed pea, cannellini beans, carob, chickpea, clover, cowpea, English pea, fava bean, green pea, hyacinth bean, kidney bean, lentil, lima bean, lima beans, lupine, mesquite, moth bean, mung bean, navy bean, peas, pigeon pea, pinto beans, rice bean, runner bean, sweet lupine, tepary bean, winged bean, and yellow pea, or combinations thereof.

The grain substrates may be selected from the following list: Amaranth, barley, buckwheat, corn, farro, fonio, kaniwa, millet, oats, quinoa, rice, rye, sorghum, teff, triticale, and wheat.

The seed substrates may be selected from the following list: Chia seeds, cotton seed, flax, hemp, poppy, pumpkin, safflower, and sunflower, or combinations thereof.

In some embodiments, the substrate material may be further supplemented with vitamins, minerals and/or nitrogen, or combinations thereof, to enhance microbial growth and production of metabolites that impart flavor. In other aspect, the substrate material may require the addition of an agent to adjust or modify its pH to facilitate microbial growth.

In some embodiments, the method of producing nutrient-dense flour blend comprises using legumes, grains or seeds, or a combination thereof. In some variations, the substrate comprises at least 50% legume material. In one aspect, the substrate inputs are whole legumes, grains and/or seeds. In another aspect, the same plant substrate inputs are mashed, ground, or otherwise reduced in size by another method to expose a greater surface area for microbial interaction.

In some embodiments, the methods of producing nutrient-dense flour blends comprises using one or more bacterial strains, alone or in combination with one or more fungal strains.

The bacterial strains may be selected from the following genera: Bifidobacterium sp., Brevibacterium sp., Corynebacterium sp., Enterococcus sp., Lactobacillus sp., Lactiplantibacillus sp., Lactococcus sp., Leuconostoc sp., Pediococcus sp., Periweissella sp., Propionibacterium sp., and Streptococcus sp.

The fungal strains may be selected from the following genera: Agaricus sp., Aspergillus sp., Cordyceps sp., Cyberlindnera sp., Debaryomyces sp., Flammulina sp., Fusarium sp., Ganoderma sp., Geotrichum sp., Grifola sp., Hericium sp., Hypsizygus sp., Kluyveromyces sp., Lentinula sp., Monascus sp., Morchella sp., Neurospora sp., Ophiocordyceps sp., Penicillium sp., Pholiota sp., Pleurotus sp., Polyporus sp., Rhizopus sp., Rhodotorula sp., Saccharomyces sp., Trametes sp., Tuber sp., Volvariella sp., Xylaria sp., Yarrowia sp., Zygosaccharomyces sp.

In an aspect of the invention, the fungal strains may be selected from the following species: Agaricus blazei, Agaricus bisporus, Agaricus brasiliensis, Aspergillus niger, Aspergillus oryzae, Cordyceps militaris, Cyberlindnera jadinii, Debaryomyces hansenii, Flammulina velutipes, Fusarium graminearum, Fusarium venenatum, Ganoderma lucidum, Geotrichum candidum, Grifola frondosa, Hericium erinaceus, Hypsizygus marmoreus, Kluyveromyces marxianus, Lentinula edodes, Monascus purpureus, Morchella esculenta, Morchella hortensis, Neurospora crassa, Neurospora intermedia, Neurospora sitophila, Ophiocordyceps sinensis, Penicillium camemberti, Penicillium roqueforti, Pholiota nameko, Pleurotus citrinopileatus, Pleurotus djamor, Pleurotus eryngii, Pleurotus ostreatus, Polyporus umbellatus, Rhizopus arrhizus, Rhizopus microsporus, Rhizopus microsporus var. oligosporus, Rhizopus oryzae, Rhizopus stolonifer, Rhodotorula glutinis, Saccharomyces cerevisiae, Trametes versicolor, Tuber magnatum, Tuber melanosporum, Volvariella volvacea, Xylaria hypoxylon, Yarrowia lipolytica, and Zygosaccharomyces rouxii.

In some embodiments, the method of producing nutrient-dense flour blends involves adding one or more microbial seed cultures comprising one or more strains to the substrate and allowing the cultures to ferment the material. As these microbial cultures grow on the substrate, they lead to the transformation of the material by metabolizing proteins, sugars, fats, and other components, which results in the development of distinctive flavors, leading to an increase in nutrient bioavailability, and alteration of organoleptic properties. The fermentation process is carefully controlled to generate specific flavor profiles from the microbial activity. In some variations, all or part of the microbes from the initial inoculum remain active throughout the fermentation process, enhancing the flavor complexity over time. In another embodiment, after initial fermentation steps, additional microbial cultures may be added to further ferment the material and refine the flavor and nutritional content by continuing to metabolize the available nutrients.

In some aspects, the microbial cultures may be added at a concentration of 1e5, 1e6, 1e7, 1e8, or 1e9 per 1 kg of input substrate material, or an intermediate amount. In other aspects, one bacterial seed culture may first be added at a concentration of 1e6, 1e7, 1e8, 1e9 and allowed to grow on the substrate. In another aspect, a fungal seed culture may be added at a concentration of 1e6, 1e7, 1e8, or 1e9 per 1 kg of substrate input material, or an intermediate amount.

In some embodiments, the bacterial and fungal cultures are added at the same time at the beginning of the fermentation. In another aspect, the bacterial seed culture is added first, and the fungal culture is added 3 hours later, 6 hours later, 12 hours later, 18 hour later, 24 hour later, or an intermediate amount of time later. In yet another aspect, the fungal seed culture is added first and allowed to grow on the substrate. This step would allow for the production of hydrolytic enzymes to break down major plant components into intermediate products that can be used for growth by the bacterial cultures. The bacterial cultures can be added 3 hours later, 6 hours later, 12 hours later, 18 hours later, 24 hours later, or 48 hours later, or an intermediate amount of time later. Varying the inoculation ratios of the utilized strains has a significant impact on the resulting nutrient composition and flavor profile.

The plant input substrates can be fermented at any temperature that is suitable for the specific microbial cultures employed. For instance, the material might be fermented at temperatures ranging from 26-33° C. to favor the growth of mesophilic cultures, or at temperatures between 35-41° C. for optimal growth of thermophilic cultures. The fermentation process can be conducted at temperatures varying from approximately 10° C. to 50° C., based on the ideal growth temperatures for the microbes utilized.

In another embodiment, the temperature is first maintained at 28° C. for 3 hours, 6 hours, 12 hours, 18 hours, or 24 hours, and is then increased or decreased until the end of the fermentation.

In another aspect, the fermentation is stopped by raising the temperature, by applying steam or by another method known in the art. Alternatively, fermentation may be stopped by lowering the temperature. After fermentation stops, the fermented product is dehydrated by any method known in the art to yield a dried product with a moisture content between 0.5-11%. The particle size of the substrate is reduced by any known method, such as milling or grinding and can then be used to formulate food products. Milling may take place by using impact mills, ball mills, hammer mills, pin mills, vertical mills, jet mills, attrition mills, shearing mills, micronizers, and/or roller mills.

The flavor profile of the starting materials is often undesirable and is altered via the fermentation process, and then maintained or altered to preferable flavors through the downstream processing steps. Additionally, optimizing the fermentation process can help reduce any potential earthy or bitter flavors, enhancing the overall taste experience. The flavor compounds of interest can be fatty acids, volatile organic compounds, amino acids, or other chemicals known in the art to impart flavor. Note that taste perception is complex and the presence of some compounds at low ppm concentrations undetectable by modern analytical tooling can have profound effects on the perceived flavor. Scent plays an important role in flavor profile, and aldehydes, ketones, pyrazines and/or esters that may be generated during the fermentation process may impart better flavor profiles.

Example 1: The substrates for fermentation can be prepared by well-known methods in the art. In one embodiment of the present invention, 200 g of chickpeas are soaked for 8-24 hours in cold water, followed by mechanical dehulling and steaming or boiling for 20-40 minutes. After cooling to permissive temperatures, the chickpeas are inoculated with R. oligosporus and incubated at 28° C. for 6 hours. At the six hour mark, the substrate is inoculated with Lactiplantibacillus plantarum and incubated for an additional 24 hours. The fermentation is stopped by applying steam to the fermented product for 5 minutes. The product is then dehydrated in an electrical dehydrator (MagicMill) at a temperature of 45-55° C. until there is no more change in weight of the material (indicating that no more moisture can be removed). The resulting powder/flour can be stored at room temperature in an airtight container for at least three months without losing flavor.

Example 2: A mix of oats, chia, hemp, and pumpkin seeds in a ratio of 5:1.5:1.5:2 was used. Oats were cooked directly by boiling for 10-20 minutes. Chia, hemp and pumpkin were flash roasted to remove any possible surface level contaminants. The components were mixed and inoculated with only R. oligosporus and allowed to ferment for 36 hours. The resulting flavor of the flour was nutty and sweet as compared to the starting materials.

Example 3: The fermented flour from Example 1 was used to formulate a cracker snack food product. The base recipe comprises using at least 40% of the fermented flour with a standard unfermented flour, a fat, salt, and combination of seeds to impact additional texture. The dough is hydrated at least 45%, flattened by rolling, cut into typical cracker sized pieces, and baked.

Example 4: A nutritional analysis was performed. The following table 1 shows the results of that nutritional analysis. Fiber analysis was performed according to AOAC 2011.25.

TABLE 1

| per 100 g DM | cooked | fermented |
|---|---|---|
| calories | 411 | 418 |
| fat | 6.35 | 8.27 |
| total carbohydrates | 70.23 | 59.63 |
| protein | 18.31 | 26.31 |
| moisture | 1.71 | 3.6 |
| ash | 3.4 | 2.19 |
| low MW DF (SDFS) (w/w) | 3.30% | 2.20% |
| insoluble high MW DF (IDF) (also RS) | 17% | 22.10% |
| soluble high MW (SDFP) | 1.20% | 1.60% |
| total DF (TDF = IDF + SDFP + SDFS) (w/w) | 21.40% | 26.80% |

DF = dietary fiber, TDF = total dietary fiber, IDF = insoluble dietary fiber, RS = resistant starch, SDFP = soluble dietary fiber that precipitates in 78% ethanol SDFS = soluble dietary fiber that remains soluble in 78% ethanol.

In Table 1, unfermented and fermented chickpea samples were analyzed for their proximate and fiber content (according to AOAC 2011.25). Compared to the unfermented control, the fermented chickpea flour has a higher protein content, and a higher insoluble high molecular fiber, which includes resistant starch. Reference values for the raw chickpea seeds can be found in a reference by Rachwa-Rosiak et al 2015. As can be seen in Table 1, one is able to attain greater protein and fiber values with fermented chickpeas relative to the cooked (but unfermented) chickpeas.

### Example 5

Table 2 demonstrates how one sees a protein increase upon fermentation of the chickpeas. The highest protein levels were seen for samples f5 and f10 respectively.

TABLE 2

| | FDA DAILY VALUE (g)* |
|---|---|
| | 50 |
| | % DV |
| Sample/Condition | Protein |
| raw | 40.4 |
| cooked 1 | 40 |
| cooked 2 | 39.6 |
| f1 | 40 |
| f2 | 40 |
| f3 | 43.4 |
| f4 | 54.6 |
| f5 | 64.8 |
| f6 | 47.4 |
| f7 | 55.4 |
| f8 | 44.4 |
| f9 | 57.2 |
| f10 | 64 |
| f11 | 42.8 |
| f12 | 45 |
| f13 | 55 |
| f14 | 60.6 |
| f15 | 44 |
| f16 | 46 |
| f17 | 49.4 |
| f18 | 54.4 |
| f19 | 50.8 |

*Reference: US FDA Human Foods Program

### Example 6

Folate levels were also measured for some of the fermentation runs with table 3 showing the results of those folate levels.

TABLE 3

| sample | mcg/100 g | % DV | % change |
|---|---|---|---|
| f1 | 438 | 87.6 | 134 |
| f2 | 337 | 67.4 | 80 |
| f3 | 342 | 68.4 | 83 |
| f4 | 299 | 59.8 | 60 |
| unferm. control | 187 | 37.4 | n/a |

### Example 7

The results from Examples 4 and 5 show that the protein content increases upon fermentation of chickpeas. Table 4 shows the relative amino acid content for raw, cooked, and fermented chickpeas. The amino acid were analyzed post sample acid hydrolysis by reversed phase HPLC, with on-column OPA (o-phthalaldehyde) derivatization. Post fermentation the total composition of amino acids is increased by over 30%. From table 4, it can be seen that essential amino acid content (note the first eight bolded amino acids) post fermentation is increased by about 37% relative to the raw chickpea substrate. DM=Dry Matter.

TABLE 4

| | g/100 g DM | g/100 g DM | g/100 g DM |
|---|---|---|---|
| AA | Raw | Cooked | Fermented |
| Histidine | 0.45 | 0.47 | 0.62 |
| Isoleucine | 0.84 | 0.87 | 1.21 |
| Leucine | 1.49 | 1.56 | 2.08 |
| Lysine | 1.34 | 1.34 | 1.71 |
| Methionine | 0.29 | 0.29 | 0.42 |
| Phenylalanine | 1.12 | 1.17 | 1.48 |
| Threonine | 0.730 | 0.75 | 1.00 |
| Valine | 0.910 | 0.95 | 1.33 |
| Alanine | 0.87 | 0.9 | 1.26 |
| Arginine | 1.58 | 1.58 | 1.91 |
| Aspartic Acid | 2.26 | 2.31 | 2.86 |
| Cysteine | 0.32 | 0.28 | 0.43 |
| Glutamic Acid | 3.43 | 3.32 | 3.95 |
| Glycine | 0.800 | 0.81 | 1.06 |
| Proline | 0.84 | 0.87 | 1.17 |
| Serine | 1.01 | 1.05 | 1.39 |
| Tyrosine | 0.56 | 0.58 | 0.76 |
| SUM AIIAA | 18.8 | 19.1 | 24.6 |
| % Change AIIAA | — | 1.38 | 30.8 |
| SUM EAA | 7.17 | 7.4 | 9.85 |
| % Change EAA | — | 3.21 | 37.4 |

### Example 8

Raw chickpeas were milled and passed through a 60 mesh sieve. Fermented chickpea powder (see Example 1) was prepared as described previously. Samples were aliquoted and incubated at 45° C. Samples were assessed for flavor degradation as a function of storage time. The results are shown in Table 5.

TABLE 5

| day | unfermented | fermented |
|---|---|---|
| 1 | beany, grassy | nutty, toasty, aromatic |
| 9 | beany, musky | nutty, toasty |
| 20 | beany, earthy | nutty, toasty |
| 32 | earthy | nutty, toasty, biscuit |
| 45 | stale | nutty, biscuit |
| 62 | stale, earthy | nutty, sweet |

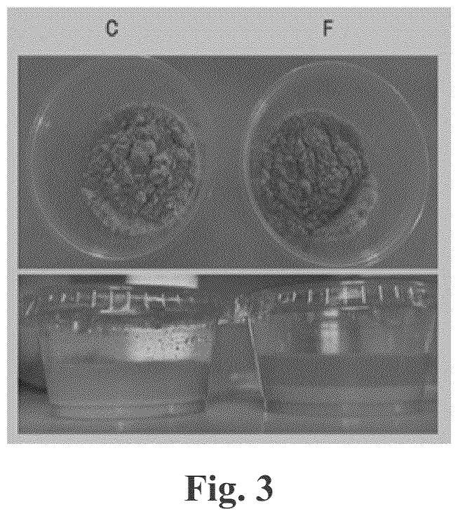

As can be seen from table 5, the fermented chickpeas had flavor that was still desirable after 32 days whereas the unfermented chickpeas had undesirable qualities from the start that went from a grassy to an earthy to a stale taste with the unfermented chickpeas being both stale and earthy at day 62. FIG. 3 shows the unfermented (i.e., C=control) from a top down view (upper left figure) and a side view (lower left figure) and the top down view (upper right figure) and the side view (bottom right figure) of the fermented (i.e., F) version adjacent to the control. It should be noted that because the fermented chickpea product appears to maintain better qualities for a longer period of time, it is believed that the fermented product is a more stable product than its unfermented comparative product.

### Example 9

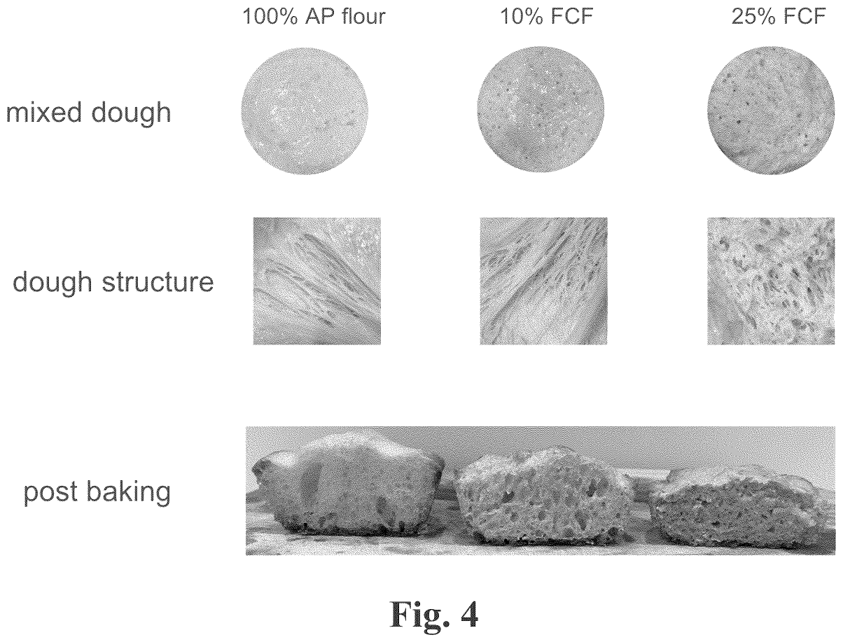

The fermented chickpea flour can be incorporated into any of a plurality of products. For example, the fermented chickpea powder can be incorporated into breads, biscuits, puddings, crackers, cakes, pancakes, crepes, waffles, nutritional bars, spreads and/or pizza doughs. Alternatively and/or additionally, the powdered flour can be used in foods that incorporate a roux. When incorporated into cakes, the chickpea flour blend powder provides a toasted and/or sweet aromatic flavor. In contrast, a control unfermented chickpea flour powder gives a nutty, bitter, and/or beany flavor. When incorporated into focaccia dough at different amounts, the dough became thinner with a higher quantity of fermented chickpea flour, with a concomitant rise in the amount of protein and the amount of folate. See FIG. 4, which shows the fermented chickpea flour in focaccia dough at concentrations of 0%, 10%, and 25% (from left to right) respectively. Table 6 shows the protein and folate levels at inclusion levels of 0%, and 10% and 25% relative to the all purpose (AP) flour only.

TABLE 6

| Inclusion Level | 0% | 10% | 25% |
|---|---|---|---|
| Protein (g) | 4.7 | 5.3 | 6 |
| Folate (mcg) | 12 | 23.5 | 41 |

Table 7 shows the nutritional content of the various focaccia recipes made with 100% all purpose flour (i.e., 100% AP), 10% fermented chickpea flour and 25% fermented chickpea flour.

TABLE 7

| | per 100 grams | per 100 grams | per 100 grams |
|---|---|---|---|
| | 100% AP | 10% FCF | 25% FCF |
| Calories | 275 | 280 | 285 |
| Total fat | 11.5 | 12 | 12.9 |
| Cholesterol | 0 | 0 | 0 |
| Sodium | 392 | 392 | 392 |
| Total Carbohydrates | 37 | 35.8 | 34.1 |
| Dietary Fiber | 1.5 | 2.6 | 4.2 |
| Sugars | 0.1 | 0.1 | 0.1 |
| Protein | 5.2 | 6.3 | 7.9 |
| Vitamin D (mcg) | 0 | 0 | 0 |
| Calcium (mg) | 10.1 | 14.2 | 20.3 |
| Iron (mg) | 0.6 | 1 | 1.7 |
| Potassium (mg) | 57.1 | 54.4 | 50.3 |

Table 8 shows the various recipe amounts for the various focaccia recipes made with 100% all purpose flour (100% AP), 10% fermented chickpea flour (FCF) and 25% FCF.

TABLE 8

| | Amount (g) | Amount (g) | Amount (g) |
|---|---|---|---|
| Ingredient | 100% AP | 10% FCF | 25% FCF |
| Flour | 550 | 495 | 412.5 |
| Water | 450 | 450 | 450 |
| Olive oil | 25 | 25 | 25 |
| Yeast | 6.5 | 6.5 | 6.5 |
| Salt | 11.5 | 11.5 | 11.5 |
| FCF | 0 | 55 | 137.5 |

### Example 10

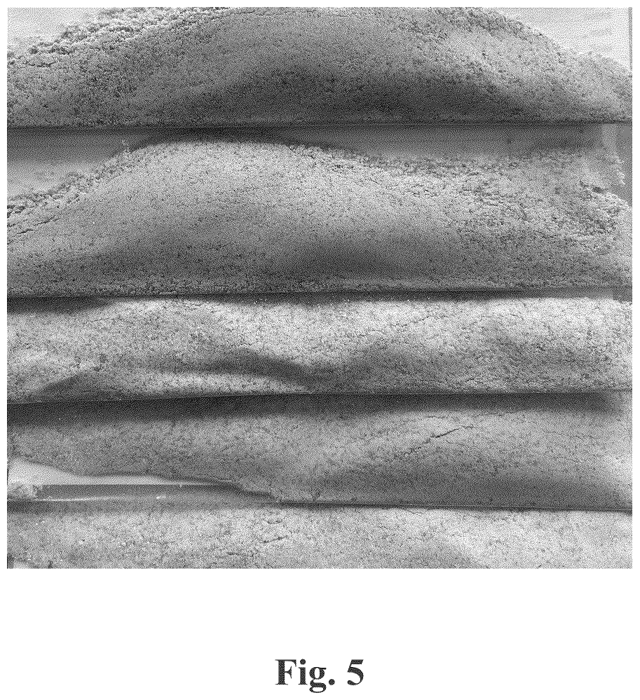

Various legumes, grains, and seeds can be fermented and powders prepared. The color and the size of the various legume blend can be appropriately adjusted, which are a function of the fermentation parameters used. FIG. 5 shows different fermented amounts of flours that display differences in particle size and color.

### Example 11

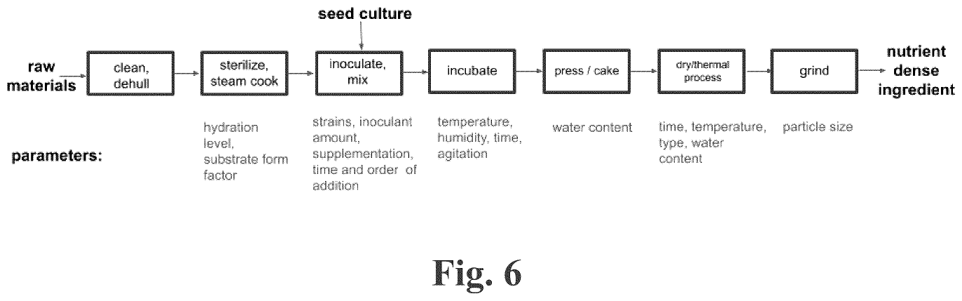

The process for generating a nutrient rich fermented product of the present invention involves a number of steps. First, one must acquire the raw materials to make the fermented product, the raw material being a legume, grain, seed and an appropriate bacteria or fungus. The legume, grain, or seed raw material is cleaned and dehulled, if necessary. The raw material can be further cleaned by sterilizing using steam or another methodology. Subsequently, the steamed raw material is cooled and ready for inoculation with the appropriate fungus or bacteria. After inoculation, the mix is incubated for a period of time (e.g., at 25-30° C. for more than a day), wherein the raw material is allowed to ferment. Subsequently, the incubated raw material is pressed into a cake and a drying process occurs to remove residual moisture. The pressed and dry cake is then ground (for example, by a micronizer) to generate a nutrient-rich powder ingredient. M any of the parameters need to be adjusted and/or monitored during the process including hydration levels, strain types, humidity, agitation, time and temperature, water content, and particle size. By varying the various parameters, modifications can be attained in the final nutrient-dense product that is made. FIG. 6 shows schematically the process of going from the raw material to the nutrient dense ingredient with the final product being a powder. Some of the parameters that must be considered at each step of the process are shown below the step. In an embodiment, the fungal and bacterial strains are grown on hydrated plant material substrates, such as pulses (e.g., chickpeas, lentils, dry peas and beans). In a variation, the downstream processing comprises harvesting the entirety of the fermented biomass. After drying and milling (grinding), the resulting flour can be used to generate any of a number of food products that are enriched in nutrients relative to non-fermented flours.

In an embodiment, the present invention relates to products containing and methods of using fermented legumes, seeds, and/or grains. In a variation, the product is a food product that possesses good stability, good flavor, and high nutrient content, including protein and fiber levels that exceed products that are the same but made with the unfermented legumes seeds and/or grains.

In an embodiment, the present invention relates to a method of producing a fermented product, the method comprising:
a. providing a substrate plant material of a type selected from the group consisting of a legume, grain, seed, and combinations thereof;
b. placing the substrate plant material in a container with a set of cultures selected from the group consisting of a bacterial culture, a fungal culture, and combinations thereof to generate an inoculated substrate;
c. placing the inoculated substrate in an environment having a controlled temperature for a duration sufficient to produce the fermented product;
d, wherein the fungal culture is selected from the fungal group consisting of: Agaricus sp., Aspergillus sp., Cordyceps sp., Cyberlindnera sp., Debaryomyces sp., Flammulina sp., Fusarium sp., Ganoderma sp., Geotrichum sp., Grifola sp., Hericium sp., Hypsizygus sp., Kluyveromyces sp., Lentinula sp., Monascus sp., Morchella sp., Neurospora sp., Ophiocordyceps sp., Penicillium sp., Pholiota sp., Pleurotus sp., Polyporus sp., Rhizopus sp., Rhodotorula sp., Saccharomyces sp., Trametes sp., Tuber sp., Volvariella sp., Xylaria sp., Yarrowia sp., Zygosaccharomyces sp. and combinations thereof;
e, wherein the bacterial culture is selected from the bacterial group consisting of: Bifidobacterium sp., Brevibacterium sp., Corynebacterium sp., Enterococcus sp., Lactobacillus sp., Lactiplantibacillus sp., Lactococcus sp., Leuconostoc sp., Pediococcus sp., Periweissella sp., Propionibacterium sp., Streptococcus sp., and combinations thereof.

In a variation, the legume is selected from the group consisting of: Adzuki bean, alfalfa, Bambara groundnut, black gram, black-eyed pea, cannellini beans, carob, chickpea, clover, cowpea, English pea, fava bean, green pea, hyacinth bean, kidney bean, lentil, lima bean, lima beans, lupine, mesquite, moth bean, mung bean, navy bean, peas, pigeon pea, pinto beans, rice bean, runner bean, sweet lupine, tepary bean, winged bean, and yellow pea. In a variation, the legume is chickpea.

In a variation, the fermented product is used to make a food product. In a variation, the food product is a bread, a biscuit, pizza dough, a cracker, a pudding, a cake, pancakes, crepes, waffles, a nutritional bar, a spread or a food product that comprises a roux.

In a variation, the seed is selected from the group consisting of: chia seeds, cotton seed, flax, hemp, poppy, pumpkin, safflower, sunflower seeds, and combinations thereof.

In a variation, the grain is selected from the group consisting of: Amaranth, barley, buckwheat, corn, farro, fonio, kaniwa, millet, oats, quinoa, rice, rye, sorghum, teff, triticale, wheat, and combinations thereof.

In a variation, a protein content is higher in the fermented product comprising the legume, grain, seed, or combination thereof relative to a product that incorporates the legume, grain, seed, or combination thereof that has not been fermented. In a variation, the protein content in the fermented product is at least 20% more relative to the product that incorporates the legume, grain, seed, or combination thereof that has not been fermented. In a variation, the protein content in the fermented product is at least 30% more relative to the product that incorporates the legume, grain, seed, or combination thereof that has not been fermented.

In a variation, the protein content in the fermented product has a protein content that is greater in at least two of histidine, isoleucine, leucine, lysine, methionine, phenylalanine, threonine, and valine. In a variation, the fermented product has a protein content that is greater in at least three of histidine, isoleucine, leucine, lysine, methionine, phenylalanine, threonine, and valine. In a variation, the fermented product has a protein content that is greater in at least four of histidine, isoleucine, leucine, lysine, methionine, phenylalanine, threonine, and valine.

In an embodiment, the present invention relates to a food product, the food product being made by using a fermented product, the fermented product being a substrate plant material of a type selected from the group consisting of a legume, grain, seed, and combinations thereof, wherein the fermented product is made by a process that comprises fermenting the substrate plant material with a set of cultures selected from the group consisting of a bacterial culture, a fungal culture, and combinations thereof. In a variation, the food product has a higher protein content than a product that is identical to the food product made by using the fermented product with the exception of the fermented product. In a variation, the fungal culture is selected from the fungal group consisting of: Agaricus sp., Aspergillus sp., Cordyceps sp., Cyberlindnera sp., Debaryomyces sp., Flammulina sp., Fusarium sp., Ganoderma sp., Geotrichum sp., Grifola sp., Hericium sp., Hypsizygus sp., Kluyveromyces sp., Lentinula sp., Monascus sp., Morchella sp., Neurospora sp., Ophiocordyceps sp., Penicillium sp., Pholiota sp., Pleurotus sp., Polyporus sp., Rhizopus sp., Rhodotorula sp., Saccharomyces sp., Trametes sp., Tuber sp., Volvariella sp., Xylaria sp., Yarrowia sp., Zygosaccharomyces sp. and combinations thereof.

In a variation, the bacterial culture is selected from the bacterial group consisting of: Bifidobacterium sp., Brevibacterium sp., Corynebacterium sp., Enterococcus sp., Lactobacillus sp., Lactiplantibacillus sp., Lactococcus sp., Leuconostoc sp., Pediococcus sp., Periweissella sp., Propionibacterium sp., Streptococcus sp., and combinations thereof.

In a variation, the fermenting of the substrate plant material is performed at a constant temperature of 28° C. to 30° C. for a period of at least 30 hours. In a variation, the food product comprises a bread, a biscuit, pizza dough, a cracker, a pudding, a cake, pancakes, crepes, waffles, nutritional bars, spreads, or a food product that comprises a roux. In a variation, the seed is selected from the group consisting of: chia seeds, cotton seed, flax, hemp, poppy, pumpkin, safflower, sunflower seeds, and combinations thereof, and the grain is selected from the group consisting of: Amaranth, barley, buckwheat, corn, farro, fonio, kaniwa, millet, oats, quinoa, rice, rye, sorghum, teff, triticale, wheat, and combinations thereof. In a variation, the legume is selected from the group consisting of: Adzuki bean, alfalfa, Bambara groundnut, black gram, black-eyed pea, cannellini beans, carob, chickpea, clover, cowpea, English pea, fava bean, green pea, hyacinth bean, kidney bean, lentil, lima bean, lima beans, lupine, mesquite, moth bean, mung bean, navy bean, peas, pigeon pea, pinto beans, rice bean, runner bean, sweet lupine, tepary bean, winged bean, and yellow pea. In a variation, the legume is chickpea.

All references cited herein are incorporated by reference in their entireties for all purposes.
Lane, Melissa M., Elizabeth Gamage, Shutong Du, Deborah N. Ashtree, Amelia J. McGuinness, Sarah Gauci, Phillip Baker, et al. 2024. “Ultra-Processed Food Exposure and Adverse Health Outcomes: Umbrella Review of Epidemiological M eta-Analyses.” BMJ 384 (February):e077310.
Siciliano, Rosa Anna, Anna Reale, Maria Fiorella Mazzeo, Stefano Morandi, Tiziana Silvetti, and Milena Brasca. 2021. “Paraprobiotics: A New Perspective for Functional Foods and Nutraceuticals.” Nutrients 13 (4). https://doi.org/10.3390/nu13041225.
Teoh, Sze Qi, Nyuk Ling Chin, Chun Wie Chong, Adiratna Mat Ripen, Syahmeer How, and Joyce Jen Li Lim. 2024. “A Review on Health Benefits and Processing of Tempeh with Outlines on Its Functional Microbes.” Future Foods 9 (June):100330.
Human Foods Program. 2024. “Daily Value on the Nutrition and Supplement Facts Labels.” U.S. Food and Drug Administration. FDA. Sep. 9, 2024. https://www.fda.gov/food/nutrition-facts-label/daily-value-nutrition-and-supplement-facts-labels.
Rachwa-Rosiak, Danuta, Ewa Nebesny, and Grażyna Budryn. 2015. “Chickpeas—composition, Nutritional Value, Health Benefits, Application to Bread and Snacks: A Review.” Critical Reviews in Food Science and Nutrition 55 (8): 1137-45.

It should be understood and it is contemplated and within the scope of the present invention that any feature that is enumerated above can be combined with any other feature that is enumerated above as long as those features are not incompatible. Whenever ranges are mentioned, any real number that fits within the range of that range is contemplated as an endpoint to generate subranges. In any event, the invention is defined by the below claims.

What is claimed is:

1. A method of producing a fermented product, the method comprising:
a. providing a substrate plant material of a type selected from the group consisting of a legume, grain, seed, and combinations thereof;
b. placing the substrate plant material in a container with a set of cultures selected from the group consisting of a bacterial culture, a fungal culture, and combinations thereof to generate an inoculated substrate;
c. placing the inoculated substrate in an environment having a controlled temperature for a duration sufficient to produce the fermented product;
d, wherein the fungal culture is selected from the fungal group consisting of: Agaricus sp., Aspergillus sp., Cordyceps sp., Cyberlindnera sp., Debaryomyces sp., Flammulina sp., Fusarium sp., Ganoderma sp., Geotrichum sp., Grifola sp., Hericium sp., Hypsizygus sp., Kluyveromyces sp., Lentinula sp., Monascus sp., Morchella sp., Neurospora sp., Ophiocordyceps sp., Penicillium sp., Pholiota sp., Pleurotus sp., Polyporus sp., Rhizopus sp., Rhodotorula sp., Saccharomyces sp., Trametes sp., Tuber sp., Volvariella sp., Xylaria sp., Yarrowia sp., Zygosaccharomyces sp. and combinations thereof;
e. wherein the bacterial culture is selected from the bacterial group consisting of: Bifidobacterium sp., Brevibacterium sp., Corynebacterium sp., Enterococcus sp., Lactobacillus sp., Lactiplantibacillus sp., Lactococcus sp., Leuconostoc sp., Pediococcus sp., Periweissella sp., Propionibacterium sp., Streptococcus sp., and combinations thereof.

2. The method of claim 1, wherein the legume is selected from the group consisting of: Adzuki bean, alfalfa, Bambara groundnut, black gram, black-eyed pea, cannellini beans, carob, chickpea, clover, cowpea, English pea, fava bean, green pea, hyacinth bean, kidney bean, lentil, lima bean, lima beans, lupine, mesquite, moth bean, mung bean, navy bean, peas, pigeon pea, pinto beans, rice bean, runner bean, sweet lupine, tepary bean, winged bean, and yellow pea.

3. The method of claim 2, wherein the legume is chickpea.

4. The method of claim 1, wherein the fermented product is used to make a food product.

5. The method of claim 4, wherein the food product is a bread, a biscuit, pizza dough, a cracker, a pudding, a cake, pancakes, crepes, waffles, a nutritional bar, a spread, or a food product that comprises a roux.

6. The method of any one of claim 1, wherein the seed is selected from the group consisting of: chia seeds, cotton seed, flax, hemp, poppy, pumpkin, safflower, sunflower seeds, and combinations thereof.

7. The method of any one of claim 1, wherein the grain is selected from the group consisting of: Amaranth, barley, buckwheat, corn, farro, fonio, kaniwa, millet, oats, quinoa, rice, rye, sorghum, teff, triticale, wheat, and combinations thereof.

8. The method of claim 1, wherein a protein content is higher in the fermented product comprising the legume, grain, seed, or combination thereof relative to a product that incorporates the legume, grain, seed, or combination thereof that has not been fermented.

9. The method of claim 8, wherein the protein content in the fermented product is at least 20% more relative to the product that incorporates the legume, grain, seed, or combination thereof that has not been fermented.

10. The method of claim 9, wherein the protein content in the fermented product is at least 30% more relative to the product that incorporates the legume, grain, seed, or combination thereof that has not been fermented.

11. The method of claim 10, wherein the protein content in the fermented product has a protein content that is greater in at least two of histidine, isoleucine, leucine, lysine, methionine, phenylalanine, threonine, and valine.

12. A food product, said food product made by using a fermented product, the fermented product being a substrate plant material of a type selected from the group consisting of a legume, grain, seed, and combinations thereof, wherein the fermented product is made by a process that comprises fermenting the substrate plant material with a set of cultures selected from the group consisting of a bacterial culture, a fungal culture, and combinations thereof.

13. The food product of claim 12, wherein the food product has a higher protein content than a product that is identical to the food product made by using the fermented product with the exception of the fermented product.

14. The food product of claim 12, wherein the fungal culture is selected from the fungal group consisting of: Agaricus sp., Aspergillus sp., Cordyceps sp., Cyberlindnera sp., Debaryomyces sp., Flammulina sp., Fusarium sp., Ganoderma sp., Geotrichum sp., Grifola sp., Hericium sp., Hypsizygussp., Kluyveromyces sp., Lentinula sp., Monascus sp., Morchella sp., Neurospora sp., Ophiocordyceps sp., Penicillium sp., Pholiota sp., Pleurotus sp., Polyporus sp., Rhizopus sp., Rhodotorula sp., Saccharomyces sp., Trametes sp., Tuber sp., Volvariella sp., Xylaria sp., Yarrowia sp., Zygosaccharomyces sp. and combinations thereof.

15. The food product of claim 12, wherein the bacterial culture is selected from the bacterial group consisting of: Bifidobacterium sp., Brevibacterium sp., Corynebacterium sp., Enterococcus sp., Lactobacillus sp., Lactiplantibacillus sp., Lactococcus sp., Leuconostoc sp., Pediococcus sp., Periweissella sp., Propionibacterium sp., Streptococcus sp., and combinations thereof.

16. The food product of claim 12, wherein fermenting the substrate plant material is performed at a constant temperature of 28° C. to 30° C. for a period of at least 30 hours.

17. The food product of claim 12, wherein the food product comprises a bread, a biscuit, pizza dough, a cracker, a pudding, a cake, pancakes, crepes, waffles, a nutritional bar, a spread, or a food product that comprises a roux.

18. The food product of claim 12, wherein the seed is selected from the group consisting of: chia seeds, cotton seed, flax, hemp, poppy, pumpkin, safflower, sunflower seeds, and combinations thereof, and the grain is selected from the group consisting of: Amaranth, barley, buckwheat, corn, farro, fonio, kaniwa, millet, oats, quinoa, rice, rye, sorghum, teff, triticale, wheat, and combinations thereof.

19. The food product of claim 12, wherein the legume is selected from the group consisting of: Adzuki bean, alfalfa, Bambara groundnut, black gram, black-eyed pea, cannellini beans, carob, chickpea, clover, cowpea, English pea, fava bean, green pea, hyacinth bean, kidney bean, lentil, lima bean, lima beans, lupine, mesquite, moth bean, mung bean, navy bean, peas, pigeon pea, pinto beans, rice bean, runner bean, sweet lupine, tepary bean, winged bean, and yellow pea.

20. The food product of 19, wherein the legume is chickpea.
